(19) United States
(12) Patent Application Publication
(10) Pub. No.: US 2026/0125654 A1
(43) Pub. Date: May 7, 2026

(54) Method for Producing a Three-Dimensional Human Multiple-Myeloma Model

(71) Applicants: Etablissement Français du Sang, La Plaine Saint Denis (FR); INSTITUT NATIONAL DE LA SANTE ET DE LA RECHERCHE MEDICALE, Paris (FR); UNIVERSITE TOULOUSE III-Paul Sabatier, Toulouse Cedex 9 (FR); Centre National de la Recherche Scientifique, Paris (FR); Centre Hospitalier Universitaire de Toulouse, Toulouse (FR)

(72) Inventors: Nicolas Lucien ESPAGNOLLE, Lapeyrouse Fossat (FR); Mélanie Alexandra GADELORGE, Lagardelle Sur Lèze (FR); Jill CORRE, Toulouse (FR)

(21) Appl. No.: 19/116,223

(22) PCT Filed: Sep. 29, 2023
(86) PCT No.: PCT/EP2023/077114

§ 371 Date: Mar. 27, 2025

(30) Foreign Application Priority Data
Sep. 30, 2022 (FR) 22 09990

(51) Int. Cl. C12N 5/09

(52) U.S. Cl. CPC C12N 5/0694; C12N 2502/1352; C12N 2502/28; C12N 2503/02; C12N 2513/00

(57) ABSTRACT

The present invention relates to the method for producing a three-dimensional (3D) model of multiple myeloma (MM), in the form of spheroids, by co-culturing mesenchymal stem/stromal cells, endothelial progenitors and primary plasma cells of patient(s) affected by MM. The present invention further relates to the spheroids obtained by said method and the uses thereof.

The present invention relates to the method for producing a three-dimensional (3D) model of multiple-myeloma (MM), in the form of spheroids, by co-culturing mesenchymal stem/stromal cells, endothelial progenitors and primary plasma cells from patient(s) affected by MM. The present invention further relates to the spheroids obtained by said method and the uses thereof.

Multiple myeloma (MM) is a hematologic malignancy, also known as bone marrow cancer. MM is characterized by the excessive proliferation in the bone marrow of a type of white blood cell, the plasma cell, which has become abnormal. Plasma cells are cells of the immune system derived from the bone marrow (BM) that produce antibodies to protect the body against external attacks (bacteria, viruses). During development, genetic anomalies (deletion, chromosomal translocation) may occur, thereby transforming healthy plasma cells into malignant plasma cells. In the normal state, the plasma cells circulate in the blood while in the pathology of MM same return to the bone marrow where same cause damage at several levels.

Multiple myeloma remains an incurable disease despite recent remarkable therapeutic advances. Care currently prevents or relieves symptoms and complications, destroying pathological plasma cells and slowing the progression of the disease.

To date, multiple myeloma suffers from the absence of relevant preclinical models. Indeed, murine models are expensive, time consuming, and not representative of human pathology. Standard two-dimensional (2D) models do not provide the viability of primary plasma cells of patients and favor the use of immortalized cell lines, which makes the models too far from the pathophysiology of MDM disease

The reproduction of adult human ex vivo bone marrow is increasingly described in the literature in order to overcome animal models that are expensive, time consuming and dependent on the species barrier. Studies have begun to reveal 3D models of human ex vivo bone marrow grouping mesenchymal and vascular compartments, generally from cells derived from immortalized cell lines. For example, the vascular compartment, playing an active role in the proliferation of hematopoietic stem cells (HSCs), is often incorporated via HUVEC endothelial immortalized cell lines. Other models require a step in mice for vascularization or functional approaches. Furthermore, the short half-life of the plasma cells does not allow same to be incorporated there in an autologous manner into the current 3D models and forces same to be ignored or tumor immortalized cell lines of plasma cells to be added, which does lead to a relevant response of the model with respect to the patient.

The inventors have created a new human 3D tissue model of multiple myeloma. Same comprises the mesenchymal compartment, the vascular compartment and the plasma cells, obtained from samples from patient(s) suffering from multiple myeloma, without the use of any immortalized line cell. In particular, the maintenance of the viability of plasma cells in co-culture over more than 14 days has been resolved. As a result, a fully human preclinical ex vivo model of multiple myeloma is generated, which is genetically relevant to said patient and includes the mesenchymal, vascular and plasma cell compartments in spheroid form.

Such model makes it possible to envisage an advance in personalized medicine by means of the rapid construction of a model representative of the tumor tissues of the bone marrow of each patient.

### DETAILED DESCRIPTION OF THE INVENTION

Method for producing human multiple myeloma spheroids

The invention relates to a method for producing human multiple myeloma (MM) spheroids, comprising:
a. Culturing mesenchymal stem/stromal cells (MSCs), endothelial cells and endothelial progenitors in a culture medium;
b. Harvesting cultured MSCs, endothelial cells and endothelial progenitors; and
C. Co-culturing MSCs, endothelial cells and endothelial progenitors harvested with CD138+ primary plasma cells from a patient with MM under conditions that lead to spheroid formation. “Mesenchymal stem cells”, also called “mesenchymal stromal cells”, mean stem cells of mesodermal origin. Same are characterized phenotypically by the co-expression of a certain number of markers such as e.g. CD73, CD90, CD105, CD146, and the absence of expression of other markers, more particularly CD45, CD31 and CD34. Same can be derived from bone marrow, adipose tissue, or umbilical cord blood. Mesenchymal stem or stromal cells are of human origin and come from a patient with MM or from a healthy subject. In a preferential mode, the mesenchymal stem/stromal cells cultured in step a. are primary cells.

“Endothelial progenitors” refer to cells engaged in endothelial differentiation but which are not yet recognizable as endothelial cells under the microscope. Same are characterized phenotypically by the expression of a certain number of markers such as e.g. CD133, CD34, CD31, VEGFR2.

“Endothelial cells” refer to cells that are completely differentiated in the endothelial pathway and thus recognizable as endothelial cells under the microscope. Same are characterized phenotypically by the expression of a certain number of markers such as e.g. CD31, VE-CADHERINE, von Willebrand factor, VEGFR2.

Endothelial progenitors and endothelial cells have the ability to become organized into a network of endothelial cells, or vascular network, and hence to become organized in vessels.

In a preferential embodiment, the endothelial progenitors and the endothelial cells cultured in step a) are primary cells. The endothelial progenitors and endothelial cells can e.g. be obtained from mononuclear cells of the bone marrow.

In one embodiment, the MSCs, the endothelial cells and the endothelial progenitors were obtained from the same subject, i.e. from the same healthy subject or from the same patient suffering from MM. Preferably, the MSCs, the endothelial cells and the endothelial progenitors were obtained from only one or the same sample, more particularly a sample of bone marrow, from said healthy subject or patient suffering from MM.

“Primary cell” refers to a cell directly derived from a tissue and/or from a sample of cells of an individual.

“Culture” refers to the selection and multiplication of cultured cells. In one embodiment of the method, in step a), mesenchymal stem/stromal cells (MSCs), endothelial progenitors and endothelial cells are cultured together in the same culture medium and preferably in the same culture container. After extraction of the raw marrow, the cells are seeded e.g. at a density of 50,000 cells/cm² in flasks. The three cell types coexist and proliferate in such culture.

In one embodiment, the culture takes place in 2 dimensions (2D), in the form of an at least partially adherent monolayer. The culture preferably takes place until the cells undergo confluence. The culture typically lasts for 3 to 30 days, preferably for 5 to 25 days, or else for 10 to 20 days, 12 to 16 days, 13 to 15 days, about 2 weeks.

Preferably, cells are not cultured in the presence of a hydrogel or a solid support (ossified tissue or other scaffold).

“Hydrogel” refers to a gel in which the swelling agent is water. The matrix of a hydrogel is generally an array of polymers. Hydrogels include in particular Matrigel, and may be formed based on fibrin, collagen, agarose, gelatin, synthetic polymer or a mixture thereof.

Preferably, the cells are not cultured with an exogenous supply of complex biomolecules (e.g. cytokine, growth factors, hormones).

“Culture medium” refers to a medium suitable for the culture of mesenchymal stem/stromal cells, endothelial progenitors and endothelial cells. The culture medium is e.g. an RPMI medium supplemented with 10% fetal calf serum (FCS), a minimal essential medium α (MEM α), or an Endothelial cell Growth Medium 2 (EGM2, from Promocell) supplemented with 2% FCS or platelet lysate (PL). Same may be in different forms but is preferably liquid and is used for the culture of eukaryotic cells, more particularly mammalian cells and more particularly human cells.

According to the invention, the healthy subject or patient suffering from MM is a human being. According to certain embodiments, the patient has just been diagnosed with MM. In certain embodiments, the patient with MM is diagnosed with a relapse.

At the end of culture step a), the cultured MSCs, endothelial cells and endothelial progenitors are harvested. Harvesting is typically done by trypsination, then washing. Other agents that are used for the detachment of adherent cells without damage can replace trypsin.

The harvested MSCs, endothelial cells and endothelial progenitors are then co-cultured with CD138+ primary plasma cells from a patient with MM under conditions that lead to the formation of spheroids.

“Plasma cells” refer to cells of the immune system derived from bone marrow (BM) expressing the marker CD138+. In the case of MM, the plasma cells express the markers CD38+ and CD138+.

“Spheroids” refer to a grouping of cells linked to each other in the three dimensions of the space. Preferably, a spheroid comprises from 500 to 750,000 cells, or else from 1,000 to 500,000 cells. The spheroids of the composition according to the invention have a mean diameter comprised between 50 μm and 750 μm, preferably comprised between 100 μm and 500 μm.

Preferably, cells are not cultured in the presence of a hydrogel or a solid support (ossified tissue or other scaffold).

Preferably, the cells are not cultured with an exogenous supply of complex biomolecules (e.g. cytokine, growth factors, hormones, etc.).

Indeed, spheroids according to the invention are formed by self-organization of MSCs, endothelial cells and endothelial progenitors with plasma cells. Spheroids according to the invention are thus formed without any hydrogel, support or exogenous supply of complex biomolecule. Such approach makes it possible to limit the biases and get closer to what is observed in vivo. The use of hydrogel, support or exogenous supply of complex biomolecule can alter the behavior of certain products and thereby lead to an underestimation or an overestimation of the action potential of the products in vivo.

In one embodiment, CD138+ primary plasma cells come from a patient with MM which is different from the patient with MM, or from the healthy subject from which the cultured MSCs, endothelial cells and endothelial progenitors were obtained. The production method is then used to obtain heterologous human multiple myeloma (MM) spheroids. The model of heterologous human MM spheroids obtained makes it possible to combine a stroma of a patient or healthy subject with the tumor plasma cells of another patient with MM in order to study the impact of MM plasma cells on a healthy stroma and reveal therapeutic targets. On the other hand, healthy plasma cells can be associated with MM stroma in order to study the impact of MM stroma on healthy plasma cells and reveal therapeutic targets.

According to another embodiment, MSCs, endothelial cells, endothelial progenitors and CD138+ primary plasmocytes were obtained from the same patient suffering from MM. The production method then makes it possible to obtain autologous human multiple myeloma (MM) spheroids. Since bone marrow sampling is an invasive procedure, preferably, MSCs, endothelial cells, endothelial progenitors, and CD138+ primary plasma cells were obtained from a same bone marrow sample from said patient with MM.

Such embodiment has the additional difficulty of successfully conserving CD138+ primary plasma cells, and preserving the viability thereof, the time of culture of MSCs, endothelial cells, and endothelial progenitors, i.e. for 3 to 30 days of culture and generally about two weeks. Indeed, in order to be able to build autologous spheroids without resorting to a new bone marrow sample from the patient suffering from MM, it is necessary to freeze the primary CD138+ plasma cells while ensuring optimum viability of the plasma cells during the following culture of the spheroids.

Thereby, preferably, the primary CD138+ plasma cells derived from the same sample in the patient as the MSCs, endothelial cells, and endothelial progenitors, were preserved, before being co-cultured, by freezing at a temperature lower than or equal to −70° C., preferably lower than or equal to −75° C. or even −80° C., in a cryopreservation medium. The cryopreservation medium can be a medium consisting of 90% FCS+10% DMSO (v/v), or 90% of 4%+10% DMSO (v/v) human albumin solution, or commercial cryopreservation solutions such as CryoStor® CS10 (Sigma-Aldrich C2874). Preferably, the freezing of CD138+ primary plasma cells is performed within 2 h after the isolation of CD138+ primary plasma cells from the bone marrow sample.

The co-culture of MSCs, endothelial cells and endothelial progenitors with CD138+ primary plasma cells is done in ULA (Ultra-Low Adherence) plates in order to promote the formation of spheroids. CD138+ primary plasma cells and MSCs are co-cultured with a ratio, in number, of 1:1 to 4:1, preferably a ratio of about 2:1. The co-culture is carried out for 4 to 14 days, e.g. 4 to 10 days, preferably for 6 to 8 days or else about 7 days. Preferably, the co-culturing step is carried out with stirring, preferably low stirring.

The culture medium is e.g. an RPMI medium supplemented with 10% fetal calf serum (FCS), a minimal essential medium a (MEM α), or preferably an Endothelial Growth Medium 2 (EGM2, from Promocell) supplemented with 2% FCS or platelet lysate (PL). Preferably, the cells are not cultured with an exogenous supply of complex biomolecules (e.g. cytokine, growth factors, hormones, etc.).

The invention further relates to spheroids obtained or which can be obtained by the above method for producing spheroids. The human multiple myeloma (MM) spheroids include a stroma, a vascular compartment and CD138+ plasma cells from a patient with MM.

Spheroids are preferably autologous, obtained by co-culturing MSCs, endothelial cells and endothelial progenitors with CD138+ primary plasma cells from the same patient with MM, preferably coming from a same sample.

Spheroids may also be heterologous in the case where the primary CD138+ plasma cells come from a patient with MM different from the patient with MM or healthy subject from which the cultured MSCs, endothelial cells and endothelial progenitors were obtained.

### Use of the Spheroids

The subject matter of the invention is the use of autologous multiple myeloma spheroids for the selection of a therapeutic treatment suitable for the patient suffering from MM, i.e. for the selection of a therapeutic treatment to which a patient suffering from multiple myeloma (MM) is likely to respond.

Indeed, the construction of a 3D model of MM as representative as possible of the tumors of the patient makes possible a clinical follow-up and personalized medicine. Spheroid models, preferably autologous, according to the invention can be used to study the responses of the tumor tissue of the patient with MM (the patient from which the cells used to make the spheroids come from) to different treatments or combinations of treatments. Thereby, the goal is to select a therapeutic treatment to which the patient is most likely to respond.

“Treatment” or “to treat” means herein to achieve, partially or substantially, one or a plurality of the following results: partially or totally reducing the extent of the disease, improving a clinical symptom or an indicator associated with the disease, delaying, inhibiting or preventing the progression of the disease, or partially or totally delaying, inhibiting or preventing the occurrence of a relapse of the disease. “Subject “, “patient” or “ill” refers herein to a human being suffering from multiple myeloma.

### Method of Selecting a Therapeutic Treatment

The invention further comprises a method of selecting a therapeutic treatment to which a patient with multiple myeloma (MM) is likely to respond using autologous spheroids derived from said patient. The selection method comprises:
the culture of the autologous spheroids of said patient in a culture medium in the presence of at least one drug candidate for the treatment of MM, for a period of at least 3 days,
the harvesting of autologous spheroids and the dissociation thereof so as to collect the myelomatous plasma cells thereof,
analysis of the viability of collected myelomatous plasma cells; and
the selection of the at least one candidate medicinal product as a therapeutic treatment to which the patient with MM is likely to respond, on the basis of the measured viability of the collected myelomatous plasma cells.

According to one embodiment, the at least one drug candidate is selected as a therapeutic treatment to which the patient with MM is likely to respond if the measured myelomatous plasma cell viability is decreased compared to the viability of myelomatous plasma cells obtained from autologous spheroids cultured under control conditions (i.e. without the addition of drug candidate or with the addition of a control buffer), or cultured in the presence of at least one other drug candidate.

Spheroids are cultured under the same conditions as previously defined in the method for the production of autologous spheroids, except for the addition of said at least one drug candidate. According to one embodiment, the selection method includes the preparation of autologous spheroids according to the method of the invention.

MSCs, endothelial cells, endothelial progenitors and plasmocytes forming the autologous spheroids are derived from the same patient, preferably from a same bone marrow sample.

Such selection method serves to test the effectiveness of a drug candidate, or combination of drugs. The drug candidate, or the combination of drugs, is added to the spheroid culture medium between the time of formation of spheroids and up to 48 h after the formation thereof, and culture is continued for a period of at least 3 days, e.g. 4 to 10 days, preferably for 6 to 8 days or else about 7 days.

In parallel, a control culture is carried out, without the presence of drug candidate or in the presence of buffer, under the same culture conditions as in the presence of said at least one drug candidate.

Examples of medicinal products or candidate medicinal products which can be used for the treatment of MM include melphalan, lenalidomide, bortezomib, dexamethasone, C34 (compound of formula (I) as described in application WO 2018/115476 A1,

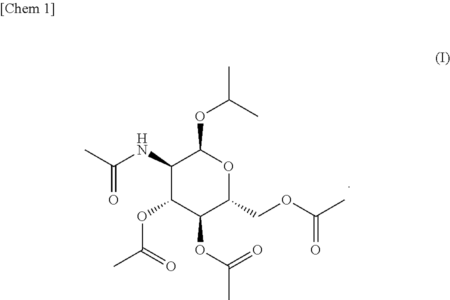 [SMILES: CC(=O)N[C@H]1[C@@H](OC(C)C)O[C@H](COC(C)=O)[C@@H](OC(C)=O)[C@@H]1OC(C)=O]

The spheroids are then harvested and dissociated mechanically, e.g. in a thermomixer and/or by repeated aspiration and displacement using a micropipette, with or without using one or a plurality of chemical agents such as trypsin, collagenase, or AccuMax dissociation solution (Capricorn Scientific GmbH). More particularly, the dissociation of the spheroids can be carried out by incubating the spheroids in a dissociation solution (comprising a protease and/or collagenase, and preferably comprising an association of protease, collagenase and DNase, e.g. AccuMax solution) with stirring (e.g. in a thermomixer at 37° C. and at 1200-1500 rpm or again about 1400 rpm, for about 10 min), then dissociating the spheroids by suction/discharge using a micropipette, and harvesting the dissociated cells (e.g. by centrifugation).

The cells are then labeled with markers to identify myelomatous plasma cells, e.g., with fluorochromes associated with specific antibodies such as CD38 to label plasma cells in particular, and CD138 to identify the myelomatous plasma cells CD38+ CD138+. The cells are then re-suspended and filtered before being analyzed by flow cytometry (FACS). The fluorochrome markers associated with specific antibodies can be e.g. CD38 FITC and CD138 AF700. The suspension and labeling solution is preferably a MACS solution and the cells are preferably filtered at 70 μm. The viability of myelomatous plasma cells is compared between different culture conditions (control condition or with at least one drug candidate), a decreased viability of myelomatous plasma cells compared to control being the sign of a promising treatment.

Suh method is faster than the use of murine models and a more relevant response due to the genetic proximity between the model and the patient, all the more when the spheroidal model is autologous. Indeed, spheroids can be generated in about 2 weeks, on average, and the selection of a treatment suitable for the patient can be made in about 1 week starting from obtaining the spheroids, which represents a total of 3 weeks, as a general rule, to be able to define a personalized treatment for the patient with MM.

### BRIEF DESCRIPTION OF FIGURES

FIG. 1 shows the viability level of MM plasma cells in heterologous spheroids following the culture thereof for 7 days with one or a plurality of drug candidates (melphalan 10 μM, lenalidomide 10 μM, C34 5 μM, melphalan 10 μM+C34 5 μM combination, lenalidomide 10 μM+C34 5 μM combination) and a control condition. On the abscissa, the MM MSCs (MM91, MM97, MM100) represent the stromal component of the spheroid. The plasmocytes MM (P64/65, P66) are the plasmocyte component of the spheroid. Each number corresponds to a given MM patient.

FIG. 2 shows the level of live MM plasma cells in spheroids following the culture thereof for 7 days with one or more drug candidates (carfilzomib 5-50 nM, pomalidomide 1-10 μM, dexamethasone 1 μM, isatuximab 1 μg/ml, daratumumab 1 μg/ml) and a control condition (NT). On the abscissa, the different treatments tested:
DCD: Daratumumab/Carfilzomib/Dexamethasone,
DPD: Daratumumab/Pomalidomide/Dexamethasone,
ICD: Isatuximab/Carfilzomib/Dexamehasone,
IPD: Isatuximab/Pomalidomide/Dexamethasone,
CD: Carfilzomib/Dexamethasone,
PD: Pomalidomide/Dexamethasone).

### EXAMPLES

### Example 1: Studies of Different Plasma Cell Freezing Protocols and Cell Viability Study

In the present example, the inventors sought to select a plasma cell freezing/thawing protocol that is the most effective for preserving the viability of the plasma cells. They studied the impact by quantifying the viability thereof after thawing by counting with trypan blue in a Malassez cell.

Following a total bone marrow sample at diagnosis in patients with multiple myeloma (MM), MM plasma cells are separated and then counted in a Malassez cell. The cells were then frozen according to one of the following protocols:
A: Buffer 90% Fetal calf serum (FCS): 10% DMSO;
Immediate freezing −80° C.
B: Buffer 90% human serum albumin (HSA): 10% DMSO;
Immediate freezing −80° C.
C: CryoStor® CS10 buffer (Sigma-Aldrich, product reference C2874);
immediate freezing−80° C.
D: CryoStor® CS10 Buffer;
Freezing −20° C. for 2 h then −80° C.
E: CryoStor® CS10 Buffer;
Freezing −20° C. for the night then −80° C.

The freezing time was on average of 31 days.

The plasma cells were then thawed and then counted with trypan blue in the Malassez cell.

TABLE 1

| | Ratio of live | Mean ratio of | |
|---|---|---|---|
| | plasma cells after | viable plasma cells | |
| | thawing to live | compared to | |
| | plasma cells | plasma cells counted | Number |
| Condition | before freezing (%) | after thawing (%) | of tests |
| A | 42.61 | 60.78 | 5 |
| B | 31.04 | 61.85 | 7 |
| C | 41.39 | 71.51 | 15 |
| D | 47.32 | 69.25 | 4 |
| E | 40.58 | 66.80 | 5 |

The results of the table hereinabove show that the current freezing conditions, namely 90% FCS or HSA+10% DMSO, appear to be less effective compared to immediate freezing conditions with CryoStor®.

### Example 2: Culture of Heterologous and Autologous Spheroids and Study of Cell Viability by Flow Cytometry

In the present example, the inventors sought to show the low impact on the viability of the plasmocytes of the steps of freezing and thawing, of culturing within the spheroids and of dissociating the spheroids before the labeling thereof. They studied the impact by quantifying the viability thereof by flow cytometry. To this end, they sought to label the cells with anti-CD38 and anti-CD138 antibodies coupled to fluorochromes in order to specifically select the MM plasma cells.

Following a total bone marrow sample at diagnosis in patients with multiple myeloma (MM), MM plasma cells are separated and then counted in a Malassez cell. The cells are then frozen in CRYOSTOR or used directly fresh for heterologous cocultures. MM total marrow cells coming from another patient were seeded according to the initial number thereof and the cells were incubated at a temperature of 37° C. and under an atmosphere with 5% carbon dioxide for about 2 weeks. Once confluence was reached, mesenchymal stem/stromal cells (MSCs), endothelial cells and endothelial progenitors were treated with trypsin and counted.

The MSC cells, the endothelial cells and the endothelial progenitors treated with trypsin and the fresh or thawed plasmocytes in a ratio of 1 MSC per 2 plasmocytes were suspended on ULA (Ultra-Low adherence) plates in 50 μL of RPMI medium (10% FCS, 1% PS). The cells were then incubated at a temperature of 37° C. and an atmosphere with 5% carbon dioxide with stirring. 150 μL of complete RPMI medium were added after incubation for 24 h and the medium was renewed 2 times per week, removing 100 μL of culture supernatant and adding 100 μL of complete RPMI medium.

Spheroid cells were mechanically dissociated at 1400 rpm with AccuMax® and then transferred into a tube suitable for flow cytometry and washed with PBS and labeled with anti-CD38 FITC and anti-CD138 AF700 antibodies in MACS buffer. The cells were then incubated at 4° C. for 30 minutes, washed and resuspended in a MACS buffer medium and filtered at 70 μm, labeled with DAPI before passing through flow cytometry.

The inventors observed that quantification of the viability of the MM plasma cells was possible with such protocol and that the viability of the MM plasma cells remained high after the manipulations of the protocol, even after 14 days of co-culture.

### Example 3: 3D Spheroids with Plasma Cells from a Patient with Multiple Myeloma and Reaction to Treatment with Melphalan

In the present example, the inventors sought to demonstrate the viability of plasma cells within spheroids as well as the accessibility of the latter for the therapeutic molecules tested.

Following a total bone marrow sample at diagnosis in patients with multiple myeloma (MM), MM plasma cells are separated and then counted in a Malassez cell. The cells were then used freshly for cocultures or were frozen in CRYOSTOR. The MM total marrow cells coming from another patient were seeded according to the initial number thereof and the cells were incubated at a temperature of 37° C. and under an atmosphere with 5% carbon dioxide for about 2 weeks. Once confluence was reached, mesenchymal stem/stromal cells (MSCs), endothelial cells and endothelial progenitors were treated with trypsin and counted.

The MSC cells, the endothelial cells and the endothelial progenitors treated with trypsin and the fresh or thawed plasmocytes in a ratio of 1 MSC per 2 plasmocytes were suspended on ULA (Ultra-Low adherence) plates in 50 μL of RPMI medium (10% FCS, 1% PS). The cells were then incubated at a temperature of 37° C. and an atmosphere with 5% carbon dioxide with stirring. 150 μL of RPMI medium were added after incubation for 24 h and the medium was renewed 2 times per week, removing 100 μL of supernatant.

A treatment with 10 μm melphalan was added to the spheroid culture medium 48 h after the formation thereof and the culture was continued for 14 days. The selection method was supplemented by a control situation, without the presence of a drug candidate. Part of the cultures was stopped after 7 (D+7) and 11 (D+11) days to analyze the viability of the plasma cells, whereas the rest of the cultures was stopped after 14 days of culture (D+14). Spheroid cells were mechanically dissociated at 1400 rpm with AccuMax® and then transferred into a tube suitable for flow cytometry and washed with PBS and labeled with the CD38 FITC and CD138 AF700 specific antibodies in MACS buffer. The cells were then incubated at 4° C. for 30 minutes, washed and resuspended in a MACS buffer medium and filtered at 70 μm, and finally labeled with the viability marker DAPI before passing through flow cytometry.

The viability of the plasma cells in untreated MM spheroids increased from 50% at D+7 and D+11 to 60% at D+14, whereas the plasma cells in MM spheroids treated with 10 μM of melphalan had a viability of 5% at D+7, less than 5% at D+11 and less than 10% at D+14.

Firstly, the inventors thus showed that the viability of the plasma cells in untreated spheroids is greater (D7, D11, D14) than in the case of 2D cultures where the primary plasma cells do not survive beyond a few days.

The inventors then showed that the localization of plasma cells within spheroids does not prevent a strong response to treatment (herein melphalan 10 μM).

### Example 4: Response of Heterologous MM Spheroids to Different Drug Candidates

Following a total bone marrow sample at diagnosis in patients with multiple myeloma (MM), MM plasma cells were separated and then counted in a Malassez cell. The cells were then used freshly or were frozen at −80° C. in CRYOSTOR. MM total marrow cells coming from the same patient of from a different patient were seeded according to the initial number thereof and the cells were incubated at a temperature of 37° C. and under an atmosphere with 5% carbon dioxide for about 2 weeks. Once confluence was reached, mesenchymal stem/stromal cells (MSCs), endothelial cells and endothelial progenitors were treated with trypsin and counted.

The MSC cells, the endothelial cells and the endothelial progenitors treated with trypsin and the fresh or thawed plasmocytes in a ratio of 1 MSC per 2 plasmocytes were suspended on ULA (Ultra-Low adherence) plates in 50 μL of RPMI medium (10% FCS, 1% PS). The cells were then incubated at a temperature of 37° C. and an atmosphere with 5% carbon dioxide with stirring. 150 μL of RPMI medium were added after incubation for 24 h and the medium was renewed 2 times per week, removing 100 μL of culture supernatant and adding 100 μL of RPMI medium.

The drug candidate, the candidate combination or the combination of candidates were added to the culture medium of spheroids between the time of the formation thereof and 48 h after the formation thereof and the culture was continued for 7 days. This selection method was completed by a control situation, without the presence of any drug candidate, of the same culture duration as in the presence of drug candidate.

The treatments tested were the following:
Melphalan 10 μM,
Lenalidomide 10 μM,
C34 5 μM
Melphalan 10 μM+C34 5 μM combination,
Lenalidomide 10 μM+C34 5 μM combination

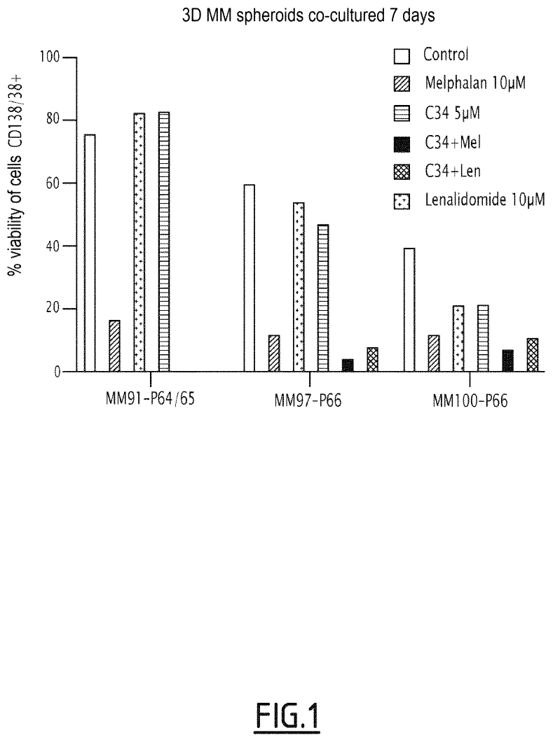

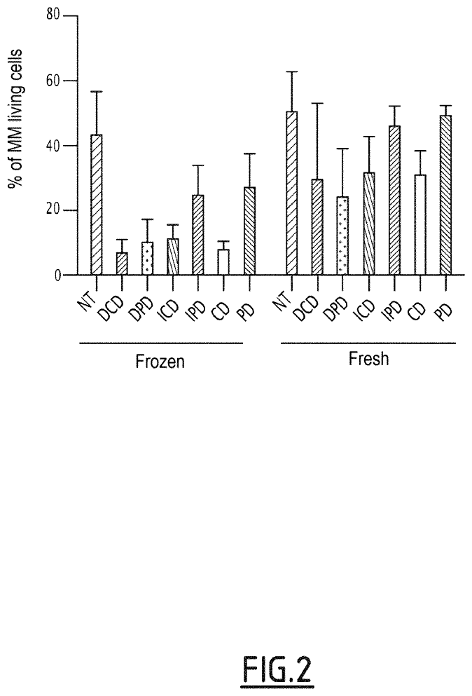

Spheroid cells were mechanically dissociated at 1400 rpm with AccuMax® and then transferred into a tube suitable for flow cytometry and washed with PBS and labeled with the CD38 FITC and CD138 AF700 specific antibodies in MACS buffer. The cells were then incubated at 4° C. for 30 minutes, washed and resuspended in a MACS buffer medium and filtered at 70 μm, and finally labeled with DAPI before passing through flow cytometry. FIG. 1 shows the viability level of MM plasma cells in heterologous spheroids following the culture thereof for 7 days with the different potential treatments tested as well as a control condition. On the abscissa, the MM MSCs (MM91, MM97, MM100) represent the stroma component of the spheroid. The plasmocytes MM (P64/65, P66) are the plasmocyte component of the spheroid. Each number corresponds to a given MM patient. FIG. 1 clearly shows the difference of behavior of the plasma cells under different conditions and the effectiveness of combined melphalan+C34 and lenalidomide+C34 treatments in decreasing the viability of MM plasma cells

### Example 5: Response of Spheroids to Different Drug Candidates and Combination of Candidates

The total marrow cells of patients with multiple myeloma (MM), were first seeded in flasks in EGM2 medium according to the initial number in the tube. The cells were incubated at 37° C. and 5% CO₂ for approximately 2 weeks.

Plasma cells from patients with multiple myeloma (MM) were first counted in Malassez cells and Trypan blue. The cells were either frozen in CryoStor (autologous culture) or used directly for heterologous spheroid coculture.

The MSCs to be used for the co-culture were trypsinated and then counted.

On a 96-well ULA plate, and in RPMI medium (10% FBS):
For each plasma cell sample recovered (fresh or thawed), a control (in triplicate) with cells alone was produced (50,000 plasma cells/well).
Each plasma cell sample was co-cultured with each trypsinized MSC sample (ratio 1:2=50,000 MSC per 100,000 plasma cells per well, 50 L/well)
The plate was incubated at 37° C. and 5% CO₂ with orbital stirring for 24 h at 73 rpm.
−150 μL of medium (with or without drug) were added to the medium before incubating the ULA plate again. The medium was changed once a week by removing 100 μL/well and adding 100 μL of new medium (with or without drug).
The culture stops at D7 and the spheroids were dissociated according to the protocol described hereinabove.

Carfilzomib was used in a concentration of 5-50 nM, pomalidomide was used in a concentration of 1-10 μM, dexamethasone was used in a concentration of 1 μM, isatuximab was used in a concentration of 1 μg/ml, daratumumab was used in a concentration of 1 μg/ml.

FIG. 2 shows the viability level of MM plasma cells in heterologous spheroids following the culture thereof for 7 days with the different treatments tested as well as a control condition (NT). The different treatments tested are indicated on the abscissa.
DCD: Daratumumab/Carfilzomib/Dexamethasone,
DPD: Daratumumab/Pomalidomide/Dexamethasone,
ICD: Isatuximab/Carfilzomib/Dexamehasone,
IPD: Isatuximab/Pomalidomide/Dexamethasone,
CD: Carfilzomib/Dexamethasone,
PD: Pomalidomide/Dexamethasone.

To the left of the graph, frozen plasma cell cells were used, each mode was repeated 4 times (n=4). To the right of the graph, fresh plasma cells were used, i.e. same were not frozen before being co-cultured to form spheroids; herein each mode was repeated 3 times (n=3).

1. A method for producing human multiple myeloma (MM) spheroids comprising:
a. Culturing mesenchymal stem/stromal cells (MSCs), endothelial cells and endothelial progenitors in a culture medium;
b. Harvesting cultured MSCs, endothelial cells and endothelial progenitors; and
c. Co-culturing MSCs, endothelial cells and endothelial progenitors harvested with CD138+ primary plasma cells from a patient with MM under conditions that lead to spheroid formation.

2. The production method according to claim 1, wherein the MSCs, endothelial cells, endothelial progenitors and CD138+ primary plasma cells were obtained from the same patient suffering from MM.

3. The production method according to claim 1- or 2, wherein the MSCs, endothelial cells, endothelial progenitors and CD138+ primary plasma cells were obtained from a same bone marrow sample from said patient with MM.

4. The production method according to claim 1, wherein the MSCs, endothelial cells and endothelial progenitors are cultured in step a. for 3 to 30 days.

5. The production method according to claim 1, wherein the CD138+ primary plasma cells were preserved, prior to co-culturing, by freezing at a temperature less than or equal to −70° C. in a cryopreservation medium.

6. The production method according to claim 1, wherein the CD138+ primary plasma cells and MSCs are co-cultured at a ratio of about 2:1.

7. The production method according to claim 1, wherein the co-culturing of MSCs, endothelial cells and endothelial progenitors with the primary plasma cells is carried out for 4 to 14 days.

8. Human multiple myeloma (MM) spheroids obtained by a production method according claim 1, comprising a stroma, a vascular compartment and CD138+ plasma cells of patient(s) affected by MM.

9. MM spheroids according to claim 8, wherein said spheroids are autologous spheroids.

10. A use of autologous multiple myeloma (MM) spheroids as defined in claim 9 for the selection of a therapeutic treatment to which a patient with MM is likely to respond.

11. A method of selecting a therapeutic treatment for which a patient with multiple myeloma (MM) is likely to respond, comprising:
i. The culture of autologous spheroids according to claim 9, in a culture medium in the presence of at least one drug candidate for the treatment of MM, for a period of at least 3 days;
ii. The harvesting of autologous spheroids and the dissociation thereof so as to collect the myelomatous plasma cells thereof present in autologous spheroids;
iii. The analysis of the viability of collected myelomatous plasma cells; and
iv. The selection of the at least one candidate medicinal product as a therapeutic treatment to which the patient with MM is likely to respond, on the basis of the measured viability of the collected myelomatous plasma cells.

12. A method of selecting a therapeutic treatment according to claim 11, wherein said at least one drug candidate is selected as a therapeutic treatment to which the patient with MM is likely to respond if the measured myelomatous plasma cell viability is decreased compared to the viability of myelomatous plasma cells obtained from autologous spheroids cultured under control conditions or cultured in the presence of at least one other drug candidate.

13. A method of selecting a therapeutic treatment according to claim 11, wherein said at least one drug candidate is added to the culture medium of the autologous spheroids between the time of spheroid formation and up to 48 h after the formation thereof, and culturing is continued for said period of at least 3 days.

14. A method of selecting a therapeutic treatment according to claim 12, wherein said at least one drug candidate is added to the culture medium of the autologous spheroids between the time of spheroid formation and up to 48 h after the formation thereof, and culturing is continued for said period of at least 3 days.

15. The production method according to claim 2, wherein the MSCs, endothelial cells, endothelial progenitors and CD138+ primary plasma cells were obtained from a same bone marrow sample from said patient with MM.
